(19) United States
(12) Patent Application Publication
(10) Pub. No.: US 2006/0082725 A1
(43) Pub. Date: Apr. 20, 2006

(54) Method and apparatus for measurement and correction of refractive power distribution data

(75) Inventors: Tatsuo Yamaguchi, Tokyo (JP); Naoyuki Maeda, Osaka (JP); Toshifumi Mihashi, Tokyo (JP); Yoko Hirohara, Tokyo (JP)

Correspondence Address: FOLEY AND LARDNER LLP;SUITE 500, 3000 K STREET NW, WASHINGTON, DC, 20007, US

(73) Assignee: KABUSHIKI KAISHA TOPCON

(21) Appl. No.: 10/525,321

(22) PCT Filed: Aug. 25, 2003
(86) PCT No.: PCT/JP03/10690

§ 371 Date: Mar. 28, 2005

(30) Foreign Application Priority Data
Aug. 29, 2002 (JP) 2002-251697

(51) Int. Cl. A61B 3/00

(52) U.S. Cl. 351/200

(57) ABSTRACT

Optical performance in a case where not only higher order aberrations but also lower order aberrations are added, are evaluated, lower order aberration quantities in which for example, a Strehi ratio is large and/or a phase shift is decreased, is calculated, and correction data, such as S, C and A, at that time is obtained, so that a result closer to a subjective value is obtained. An arithmetic part receives measurement data indicating a refractive power distribution of a subject eye and obtains lower order aberrations and higher order aberrations on the basis of the measurement data (S401, S403). The arithmetic part judges whether the higher order aberrations have a specified value or higher (S405). The arithmetic part changes, in a case where the higher order aberrations have the specified values or higher, lower order aberration quantities corresponding to the higher, lower aberrations having the specified values or higher, obtains appropriate correction data suitable for the subject eye (S407 to S417), and obtains the correction data (S419).

### TECHNICAL FIELD

The present invention relates to a correction data measurement method for measuring appropriate correction data, a measuring apparatus, and a recording medium recording a measurement program.

### BACKGROUND ART

Conventionally, as a technique for measuring ocular correction data, measurement of S (Sphere), C (Cylinder) and A (axis) by a refractometer has been carried out. Besides, recently, an eye characteristic measuring apparatus capable of measuring higher order aberrations has also been developed, and not only S, C and A on a line like, for example, a ring of φ3 mm as in a refractometer, but also S, C and A on a plane when a pupil diameter is made various sizes can be calculated from lower order aberrations. By the eye characteristic measuring apparatus like this, especially after a refraction correcting surgical operation or in an eye disease, values closer to prescription values of eyeglasses or contact lenses than the refractometer can be calculated (for example, see Japanese Patent Application No. 2001-119145, No. 2001-120002, No. 2001-119086, No. 2000-318534, etc.).

However, there is still a case where a difference occurs between the objective calculation result of a conventional eye characteristic measuring apparatus and the prescription value of eyeglasses, contact lenses, lenses or the like, and there has been a case where it is insufficient as the evaluation-of S, C and A.

### DISCLOSURE OF THE INVENTION

Accordingly, in the present invention, in a case where higher order aberration are included in a result of a measurement obtained by an eye characteristic measuring apparatus capable of measuring higher order aberrations, lower order aberrations corresponding to the time of an objective complete correction are not made corrective correction data, optical performance is evaluated by, for example, a Strehl ratio or a phase shift, such lower order aberration quantities that the Strehl ratio are high and/or the phase shift is decreased is calculated, and corrective correction data of S, C, A and the like at that time is obtained, so that more optimum correction data close to a prescription value of eyeglasses, contact lenses or the like is obtained, which is an object of the invention.

According to first solving means of the invention, a correction data measurement method comprises

a first step at which an arithmetic part receives measurement data indicating a refractive power distribution of a subject eye and obtains lower order aberrations and higher order aberrations on the basis of the measurement data,

a second step at which the arithmetic part judges whether the higher order aberrations have a specified value or higher, and

a third step at which the arithmetic part changes, in a case where the higher order aberrations have the specified value or higher, lower order aberration quantities corresponding to the higher order aberrations having the specified value or higher and obtains appropriate correction data suitable for the subject eye.

Besides, there are provided a correction data measurement program for causing a computer to execute the above respective steps, and a computer readable recording medium recording the correction data measurement program.

According to second solving means of the invention, a correction data measuring apparatus comprises an arithmetic part for obtaining an optical characteristic of a subject eye by performing a Zernike analysis on the basis of inclination angles of light fluxes obtained by a first light receiving part, wherein

the arithmetic part includes

first means for receiving measurement data indicating a refractive power distribution of the subject eye and obtaining lower order aberrations and higher order aberrations on the basis of the measurement data,

second means for judging whether the higher order aberrations have a specified value or higher, and

third means for changing, in a case where the higher order aberrations have the specified value or higher, lower order aberration quantities corresponding to the higher order aberrations having the specified value or higher and obtaining appropriate correction data suitable for the subject eye.

According to third solving means of the invention, a correction data measurement method comprises steps of:

receiving measurement data indicating a refractive power distribution of a subject eye and obtaining lower order aberrations and higher order aberrations on the basis of the measurement data,

forming a relational expression of a Strehl ratio and a phase shift (PTF) from the obtained lower order aberrations and higher order aberrations, and

changing the lower order aberration to obtain a condition under which the Strehl ratio becomes maximum and the phase shift (PTF) becomes substantially zero, and making lower order aberration quantities at that time a correction value.

### BRIEF DESCRIPTION OF THE DRAWINGS

FIG. 1 is a structural view of an optical system 100 of an eye optical characteristic measuring apparatus.

FIG. 2 is a structural view of an electrical system 200 of the eye optical characteristic measuring apparatus.

FIG. 3 is an explanatory view of a Landolt's ring.

FIG. 4 is a flowchart of an eye characteristic measurement.

FIG. 5 is a flowchart of a correction image simulation of step S107.

FIG. 6 is a flowchart of a first example of a best image condition calculation.

FIG. 7 is a flowchart of a second example of a best image condition calculation.

FIG. 8 is a view showing a display example concerning best image display—Strehl ratio optimization (pupil diameter of 4 mm).

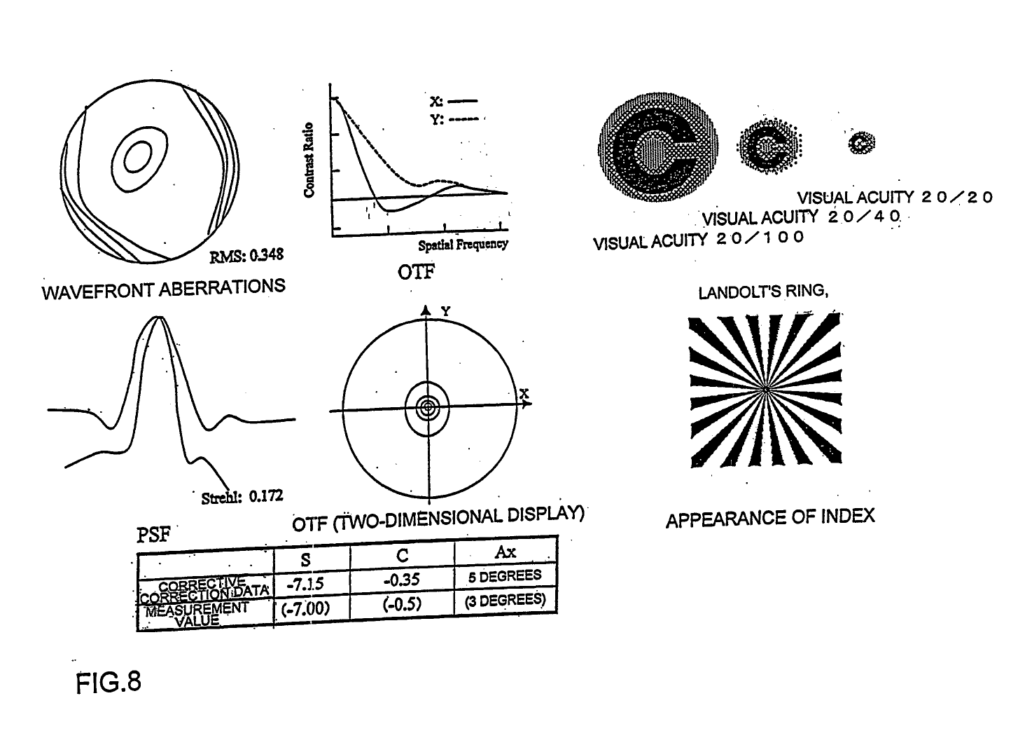

FIG. 9 is a view showing a display example concerning best image display—PTF optimization (pupil diameter of 4 mm).

FIG. 10 is a view showing a display example concerning the comparison (pupil diameter of 4 mm) of corrections added with a lower order correction and a higher order correction.

FIG. 11 is an explanatory view of an example of prescription data (pupil diameter of 4 mm) for eyeglasses or contact lenses.

FIG. 12 is an explanatory view of an example of data (pupil diameter of 4 mm) for a refraction correcting surgical operation.

FIG. 13 is a view of Zernike coefficients of (r, t) coordinates.

FIG. 14 is a view of Zernike coefficients of (x, y) coordinates.

### BEST MODE FOR CARRYING OUT THE INVENTION

1. Eye Optical Characteristic Measuring Apparatus

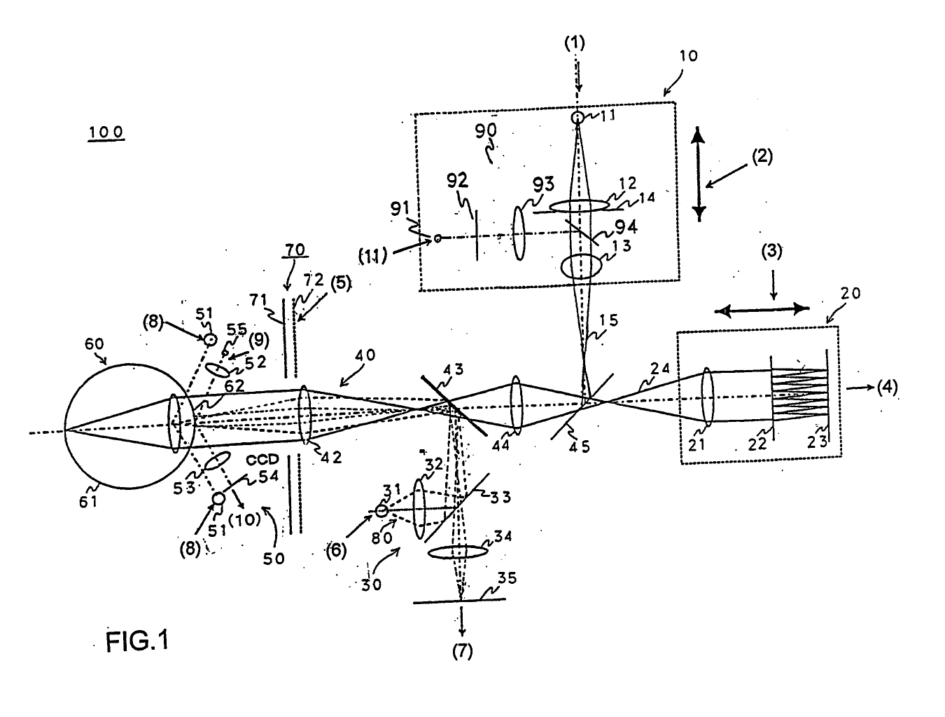

FIG. 1 is a structural view of an optical system 100 of an eye optical characteristic measuring apparatus.

The optical system 100 of the eye optical characteristic measuring apparatus is an apparatus for measuring an optical characteristic of an eye 60 to be measured as an object, and includes a first illuminating optical system 10, a first light receiving optical system 20, a second light receiving optical system 30, a common optical system 40, an adjusting optical system 50, a second illuminating optical system 70, and a second light sending optical system 80. Incidentally, with respect to the eye 60 to be measured, a retina 61 and a cornea 62 are shown in the drawing.

The first illuminating optical system 10 includes, for example, a first light source part 11 for emitting a light flux of a first wavelength, and a condensing lens 12, and is for illuminating a minute area on the retina (retina) 61 of the eye 60 to be measured with the light flux from the first light source part 11 so that its illumination condition can be suitably set. Incidentally, here, as an example, the first wavelength of the illuminating light flux emitted from the first light source part 11 is a wavelength (for example, 780 nm) of an infrared range.

Besides, it is desirable that the first light source part 11 has a high spatial coherence and a low temporal coherence. Here, the first light source part 11 is, for example, a super luminescence diode (SLD), and a point light source having high luminescence can be obtained. Incidentally, the first light source part 11 is not limited to the SLD, and for example, a laser having a high spatial coherence and a high temporal coherence can also be used by inserting a rotation diffused plate or the like to suitably lower the temporal coherence. Further, an LED having a low spatial coherence and a low temporal coherence can also be used, if light quantity is sufficient, by inserting, for example, a pinhole or the like at a position of a light source in an optical path.

The first light receiving optical system 20 includes, for example, a collimator lens 21, a Hartmann plate 22 as a conversion member for converting a part of a light flux (first light flux) reflected and returned from the retina 61 of the eye 60 to be measured into at least 17 beams, and a first light receiving part 23 for receiving the plural beams converted by the Hartmann plate 22, and is for guiding the first light flux to the first light receiving part 23. Besides, here, a CCD with little readout noise is adopted for the first light receiving part 23, and as the CCD, a suitable type of CCD, for example, a general low noise type of CCD, a cooling CCD of 1000*1000 elements for measurement, or the like is applicable.

The second illuminating optical system 70 includes a second light source 72 and a Placido's disk 71. Incidentally, the second light source 72 can be omitted. The Placido's disk (PLACIDO'S DISK) 71 is for projecting an index of a pattern composed of plural co-axial rings. Incidentally, the index of the pattern composed of the plural co-axial rings is an example of an index of a specified pattern, and a different suitable pattern can be used. Then, after an alignment adjustment described later is completed, the index of the pattern composed of the plural co-axial rings can be projected.

The second light sending optical system 80 is for mainly performing, for example, the alignment adjustment described later, and measurement and adjustment of a coordinate origin and a coordinate axis, and includes a second light source part 31 for emitting a light flux of a second wavelength, a condensing lens 32, and a beam splitter 33.

The second light receiving optical system 30 includes a condensing lens 34 and a second light receiving part 35. The second light receiving optical system 30 guides a light flux (second light flux), which is originated from the pattern of the Placido's disk 71 illuminated from the second illuminating optical system. 70 and is reflected and returned from the anterior eye part or the cornea 62 of the eye 60 to be measured, to the second light receiving part 35. Besides, it can also guide a light flux, which is emitted from the second light source part 31 and is reflected and returned from the cornea 62 of the eye 60 to be measured, to the second light receiving part 35. Incidentally, as the second wavelength of the light flux emitted from the second light source part 31, for example, a wavelength different from the first wavelength (here, 780 nm) and longer (for example, 940 nm) than that can be selected.

The common optical system 40 is disposed on an optical axis of the light flux emitted from the first illuminating optical system 10, can be included in the first and the second illuminating optical systems 10 and 70, the first and the second light receiving optical systems 20 and 30, the second light sending optical system 80 and the like in common, and includes, for example, an a focal lens 42, beam splitters 43 and 45, and a condensing lens 44. The beam splitter 43 is formed of such a mirror (for example, a dichroic mirror) that the wavelength of the second light source part 31 is sent (reflected) to the eye 60 to be measured, and the second light flux reflected and returned from the retina 61 of the eye 60 to be measured is reflected, and on the other hand, the wavelength of the first light source part 11 is transmitted. The beam splitter 45 is formed of such a mirror (for example, a polarization beam splitter) that the light flux of the first light source part 11 is sent (reflected) to the eye 60 to be measured, and the first light flux reflected and returned from the retina 61 of the eye 60 to be measured is transmitted. By the beam splitters 43 and 45, the first and the second light fluxes do not mutually enter the other optical systems to generate noise.

The adjusting optical system 50 is for mainly performing, for example, a working distance adjustment described later, includes a third light source part 51, a fourth light source part 55, condensing lenses 52 and 53, and a third light receiving part 54, and is for mainly performing the working distance adjustment.

The third illuminating optical system 90 includes an optical path for projection of an index for causing, for example, fixation of the subject eye or fogging, and includes a fifth light source part (for example, a lamp) 91, a fixed index 92 and a relay lens 93. The fixed index 92 can be irradiated to the retina 61 by the light flux from the fifth light source part 91, and the subject eye 60 is made to observe its image. The fixed index 92 and the retina 61 are put in a conjugated relation by the third illuminating optical system 90.

A sixth driving part 915 is for moving, for example, the fixed index 92 of the third illuminating optical system 90, and outputs a signal (15) to not-shown suitable movement means and drives this movement means. By this, the sixth driving part 915 can perform the movement and adjustment of the fixed index 92 of the third illuminating optical system 90.

Next, the alignment adjustment will be described. The alignment adjustment is mainly carried out by the second light receiving optical system 30 and the second light sending optical system 80.

First, the light flux from the second light source part 31 illuminates the eye 60 to be measured as the object with the substantially parallel light flux through the condensing lens 32, the beam splitters 33 and 43, and the a focal lens 42. The reflected light flux reflected by the cornea 62 of the eye 60 to be measured is emitted as a divergent light flux such as is emitted from a point at the half of the radius of curvature of the cornea 62. The divergence light flux is received as a spot image by the second light receiving part 35 through the a focal lens 42, the beam splitters 43 and 33, and the condensing lens 34.

Here, in the case where the spot image on the second light receiving part 35 is outside the optical axis, the main body of the eye optical characteristic measuring apparatus is moved and adjusted vertically and horizontally, and the spot image is made to coincide with the optical axis. As stated above, when the spot image coincides with the optical axis, the alignment adjustment is completed. Incidentally, with respect to the alignment adjustment, the cornea 62 of the eye 60 to be measured is illuminated by the third light source part 51, and an image of the eye 60 to be measured obtained by this illumination is formed on the second light receiving part 35, and accordingly, this image may be used to make the pupil center coincide with the optical axis.

Next, the working distance adjustment will be described. The working distance adjustment is mainly carried out by the adjusting optical system 50.

First the working distance adjustment is carried out by, for example, irradiating the eye 60 to be measured with a parallel light flux emitted from the fourth light source part 55 and close to the optical axis, and by receiving the light reflected from the eye 60 to be measured through the condensing lenses 52 and 53 by the third light receiving part 54. Besides, in the case where the eye 60 to be measured is in a suitable working distance, a spot image from the fourth light source part 55 is formed on the optical axis of the third light receiving part 54. On the other hand, in the case where the eye 60 to be measured goes out of the suitable working distance, the spot image from the fourth light source part 55 is formed above or below the optical axis of the third light receiving part 54. Incidentally, since the third light receiving part 54 has only to be capable of detecting a change of a light flux position on the plane containing the fourth light source part 55, the optical axis and the third light receiving part 54, for example, a one-dimensional CCD arranged on this plane, a position sensing device (PSD) or the like is applicable.

Next, a positional relation between the first illuminating optical system 10 and the first light receiving optical system 20 will be described.

The beam splitter 45 is inserted in the first light receiving optical system 20, and by this beam splitter 45, the light from the first illuminating optical system 10 is sent to the eye 60 to be measured, and the reflected light from the eye 60 to be measured is transmitted. The first light receiving part 23 included in the first light receiving optical system 20 receives the light transmitted through the Hartmann plate 22 as the conversion member and generates a received light signal.

Besides, the first light source part 11 and the retina 61 of the eye 60 to be measured form a conjugated relation. The retina 61 of the eye 60 to be measured and the first light receiving part 23 are conjugate. Besides, the Hartmann plate 22 and the pupil of the eye 60 to be measured form a conjugated relation. Further, the first light receiving optical system 20 forms a substantially conjugated relation with respect to the cornea 62 as the anterior eye part of the eye 60 to be measured, the pupil, and the Hartmann plate 22. That is, the front focal point of the a focal lens 42 is substantially coincident with the cornea 62 as the anterior eye part of the eye 60 to be measured and the pupil.

Besides, the first illuminating optical system 10 and the first light receiving optical system 20 are moved together so that a signal peak according to the reflected light at the light receiving part 23 becomes maximum on the condition that the light flux from the first light source part 11 is reflected at a point on which it is condensed. Specifically, the first illuminating optical system 10 and the first light receiving optical system 20 are moved in a direction in which the signal peak at the first light receiving part 23 becomes large, and are stopped at a position where the signal peak becomes maximum. By this, the light flux from the first light source part 11 is condensed on the eye 60 to be measured.

Besides, the lens 12 converts a diffused light of the light source 11 into a parallel light. A diaphragm 14 is positioned at an optically conjugated position with respect to the pupil of the eye or the Hartmann plate 22. The diaphragm 14 has a diameter smaller than an effective range of the Hartmann plate 22, and the so-called single path aberration measurement (method in which aberrations of an eye have an influence on only the light receiving side) is established. In order to satisfy the above, the lens 13 is disposed such that the retina conjugated point of the real light beam coincides with the front focal position, and further, in order to satisfy the conjugated relation between the lens and the pupil of the eye, it is disposed such that the rear focal position coincides with the diaphragm 14.

Besides, after a light beam 15 comes to have a light path common to a light beam 24 by the beam splitter 45, it travels in the same way as the light beam 24 paraxially. However, in the single path measurement, the diameters of the light beams are different from each other, and the beam diameter of the light beam 15 is set to be rather small as compared with the light beam 24. Specifically, the beam diameter of the light beam 15 is, for example, about 1 mm at the pupil position of the eye, and the beam diameter of the light beam 24 can be about 7 mm (incidentally, in the drawing, the light beam 15 from the beam splitter 45 to the retina 61 is omitted).

Next, the Hartmann plate 22 as the conversion member will be described.

The Hartmann plate 22 included in the first light receiving optical system 20 is a wavefront conversion member for converting a reflected light flux into plural beams. Here, plural micro-Fresnel lenses disposed on a plane orthogonal to the optical axis apply in the Hartmann plate 22. Besides, in general, with respect to the measurement object part (the eye 60 to be measured) in order to measure a sphere of the eye 60 to be measured, third-order astigmatism aberrations, and other higher order aberrations, it is necessary to-perform the measurement with at least 17 beams through the eye 60 to be measured.

Besides, the micro-Fresnel lens is an optical element, and includes, for example, a ring with a height pitch for each wavelength, and a blade optimized for emission parallel to a condensing point. The micro-Fresnel lens here is subjected to, for example, 8-level optical path length variation employing a semiconductor fine working technique, and achieves a high condensing efficiency (for example, 98%).

Besides, the reflected light from the retina 61 of the eye 60 to be measured passes through the a focal lens 42 and the collimate lens 21 and is condensed on the first light receiving part 23 through the Hartmann plate 22. Accordingly, the Hartmann plate 22 includes a wavefront conversion member for converting the reflected light flux into at least 17 beams.

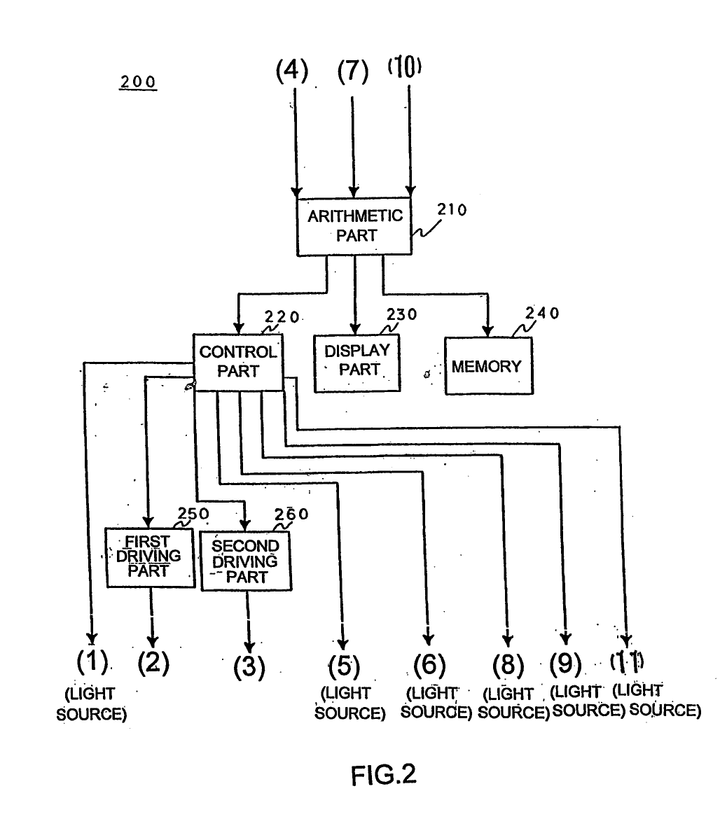

FIG. 2 is a block diagram showing an electrical system 200 of the eye optical characteristic measuring apparatus. The electrical system 200 of the eye optical characteristic measuring apparatus includes, for example, an arithmetic part 210, a control part 220, a display part 230, a memory 240, a first driving part 250, and a second driving part 260.

The arithmetic part 210 receives a received light signal (4) obtained from the first light receiving part 23, a received light signal (7) obtained from the second light receiving part 35, and a received light signal (10) obtained from the third light receiving part 54, and performs an arithmetical operation on the origin of coordinates, a coordinate axis, movement of coordinates, rotation, ocular aberrations, corneal aberrations, Zernike coefficients, aberration coefficients, a Strehl ratio, a white light MTF, a Landolt's ring pattern and the like. Besides, signals corresponding to such calculation results are outputted to the control part 220 for performing the whole control of an electric driving system, the display part 230, and the memory 240, respectively. Incidentally, the details of the arithmetic part 210 will be described later.

The control part 220 controls lighting and extinction of the first light source part 11 on the basis of the control signal from the arithmetic part 210, or controls the first driving part 250 and the second driving part 260. For example, on the basis of the signals corresponding to the operation results in the arithmetic part 210, the control part outputs a signal (1) to the first light source part 11, outputs a signal (5) to the Polacldo's disk 71, outputs a signal (6) to the second light source part 31, outputs a signal (8) to the third light source part 51, outputs a signal (9) to the fourth light source part 55, outputs a signal (11) to the fifth light source part 91, and outputs signals to the first driving part 250 and the second driving part 260.

The first driving part 250 is for moving the whole first illuminating optical system 10 in the optical axis direction on the basis of, for example, the received light signal (4) inputted to the arithmetic part 210 from the first light receiving part 23, and outputs a signal (2) to a not-shown suitable lens movement means and drives the lens movement means. By this, the first driving part 250 can perform the movement and adjustment of the first illuminating optical system 10.

The second driving part 260 is for moving the whole first light receiving optical system 20 in the optical axis direction on the basis of, for example, the received light signal (4) inputted to the arithmetic part 210 from the first light receiving part 23, and outputs a signal (3) to a not-shown suitable lens movement means, and drives the lens movement means. By this, the second driving part 260 can perform the movement and adjustment of the first light receiving optical system 20.

2. Zernike Analysis

Next, a Zernike analysis will be described. A generally known method of calculating Zernike coefficients Cᵢ^{2j-1} from Zernike polynomials will be described. The Zernike coefficients Cᵢ^{2j-i} are important parameters for grasping the optical characteristic of the subject eye 60 on the basis of inclination angles of the light fluxes obtained by the first light receiving part 23 through the Hartmann plate 22.

Wavefront aberrations W(X, Y) of the subject eye 60 are expressed using the Zernike coefficients Cᵢ^{2j-i} and the Zernike polynomials Zᵢ^{2j-i} by the following expression.

$$W ( X , Y ) = {\sum }_{i = 0}^{n} {\sum }_{j = 0}^{i} {c}_{i}^{2 j - i} {Z}_{i}^{2 j - i} ( X , Y )$$

Where, (X, Y) denotes vertical and horizontal coordinates of the Hartmann plate 22.

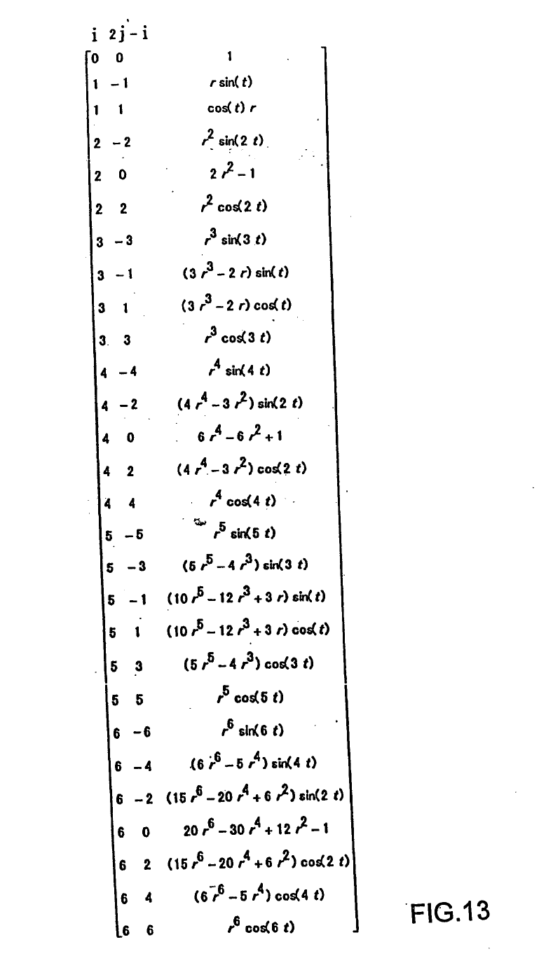

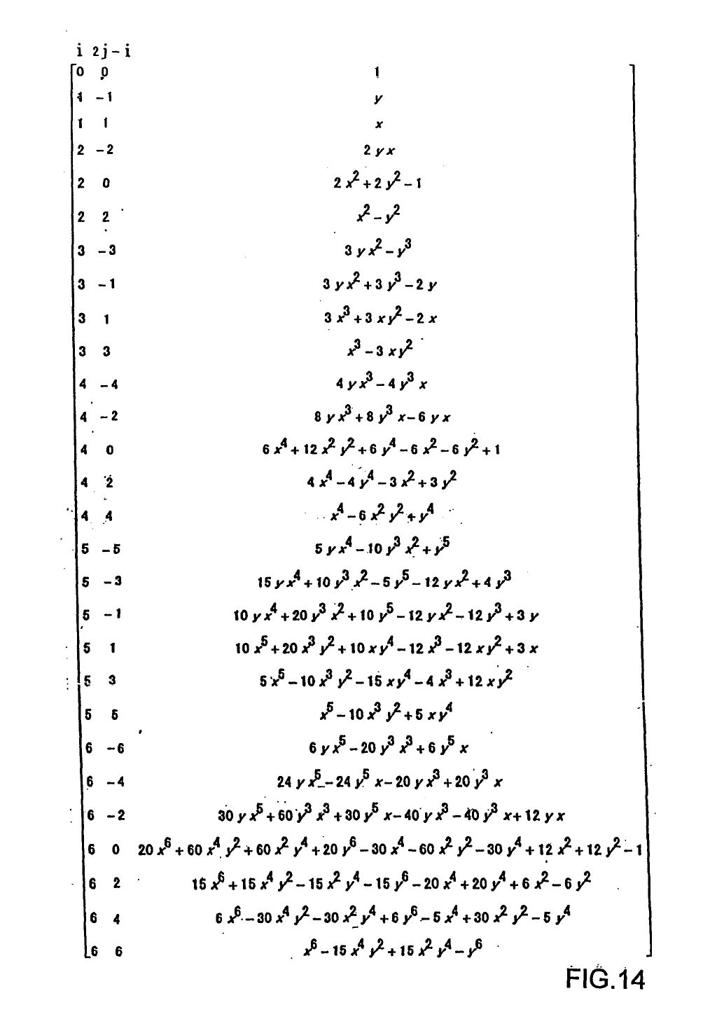

Besides, with respect to the wavefront aberrations W(X, Y), when the horizontal and vertical coordinates of the first light receiving part 23 are denoted by (x, y), a distance between the Hartmann plate 22 and the first light receiving part 23 is denoted by f, and a movement distance of a point image received by the first light receiving part 23 is denoted by (Δx, Δy), the following expression is established.

$$\frac{ϑ W ( X , Y )}{ϑ X} = \frac{Δ x}{f}$$

$$\frac{ϑ W ( X , Y )}{ϑ Y} = \frac{Δ y}{f}$$

Where, the Zernike polynomials Zᵢ^{2j-i} are expressed by the following numerical expressions. Specifically, FIG. 13 is a view of the Zernike coefficients of (r, t) coordinates, and FIG. 14 is a view of the Zernike coefficients of (x, y) coordinates.

$${Z}_{n}^{m} = {R}_{n}^{m} ( r ) \{ \frac{sin}{cos} \} \{ m θ \}$$

$$m > 0 sin$$

$$m ≦ 0 cos$$

$${R}_{n}^{m} ( r ) = {\sum }_{S = 0}^{( n - m ) / 2} {( - 1 )}^{S} \frac{( n - S ) !}{S ! \{ \frac{1}{2} ( n - m ) - S \} ! \{ \frac{1}{2} ( n + m ) - S \} !} {r}^{m}$$

Incidentally, with respect to the Zernike coefficients Cᵢ^{2j-i}, specific values can be obtained by minimizing the squared error expressed by the following numerical expression.

$$S ( x ) = {\sum }_{i = 1}^{data number} [ {\{ \frac{ϑ W ( {X}_{i} , {Y}_{i} )}{ϑ X} - \frac{Δ {x}_{i}}{f} \}}^{2} + {\{ \frac{ϑ W ( {X}_{i} , {Y}_{i} )}{ϑ Y} - \frac{Δ {y}_{i}}{f} \}}^{2} ]$$

Where, W(X, Y): wavefront aberrations, (X, Y): Hartmann plate coordinates, (Δx, Δy): a movement distance of a point image received by the first light receiving part 23, f: a distance between the Hartmann plate 22 and the first light receiving part 23, m: the number of data.

The arithmetic part 210 calculates the Zernike coefficients Cᵢ^{2j-i}, and uses this to obtain eye optical characteristics such as spherical aberrations, coma aberrations, and astigmatism aberrations.

3. Landolt's Ring

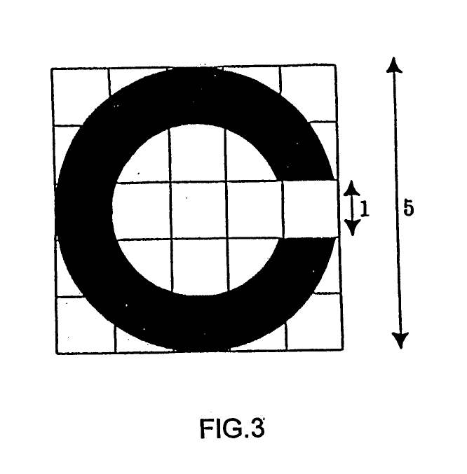

FIG. 3 is an explanatory view of a Landolt's ring.

Hereinafter, preparation of data of a luminous distribution function Land(x, y) of the Landolt's ring will be described.

The Landolt's ring is expressed by the reciprocal of a recognizable minimum visual angle, and the ability to be capable of recognizing a visual angle of one minute is called visual acuity of 20/20. For example, if the recognizable minimum visual angle is 2 minutes, the visual acuity is defined as 20/40, and if 10 minutes, the visual acuity is defined as 20/200. In general, the Landolt's ring uses, as an index, a ring in which a gap being ⅕ of the size of the outside ring is provided as shown in the drawing.

When the visual acuity is V, the size d of the Landolt's ring projected on the retina is calculated by

$$d = 5 \times 2 \cdot R tan ( \frac{1}{60 \cdot V} \times \frac{1}{2} )$$

(R: a distance between a pupil and an image point (retina))

On the basis of this expression and the definition of the Landolt's ring, a black portion of the Landolt's ring is made 0, a white portion thereof is made 1, and the luminous distribution function Land(x, y) of the Landolt's ring is prepared. The data of the prepared luminous distribution function Land(x, y) is stored in the memory 240, is read out by the arithmetic part 210, and is set correspondingly to predetermined visual acuity.

4. Correction Data Measurement Method

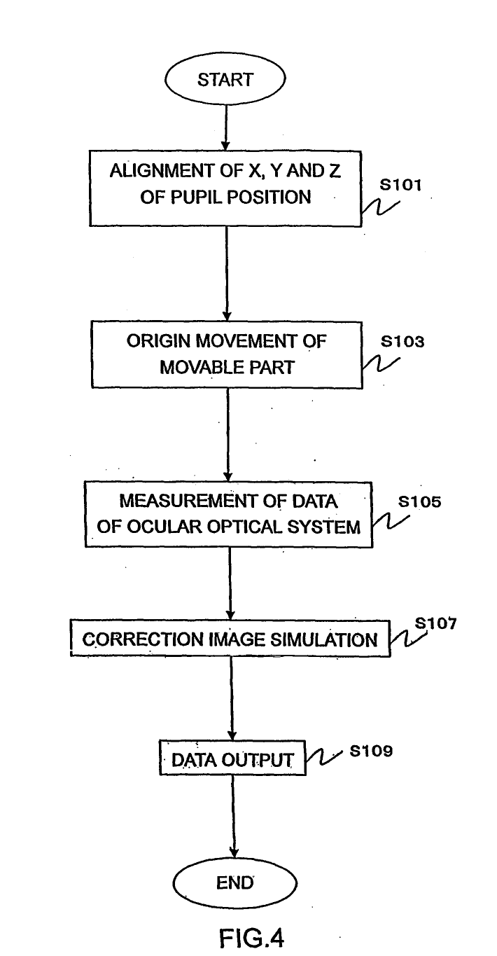

FIG. 4 is a flowchart of an eye characteristic measurement.

First, the eye characteristic measuring apparatus makes an alignment of X, Y and Z axes of the pupil position of the subject eye (S101). Next, the measuring apparatus moves the origin of a movable part (S103). For example, the Hartmann plate, the Placido's disk or the like is matched to zero diopter. The arithmetic part 210 measures the data of the ocular optical system, such as ocular aberrations or Zernike coefficients, on the basis of the measured received light signal (4), (7) and/or (10) (S105). The arithmetic part 210 performs a correction image simulation (S107). The details will be described later. The arithmetic part 210 outputs data to the correction image simulation data display part 230 and the memory 240 (S109).

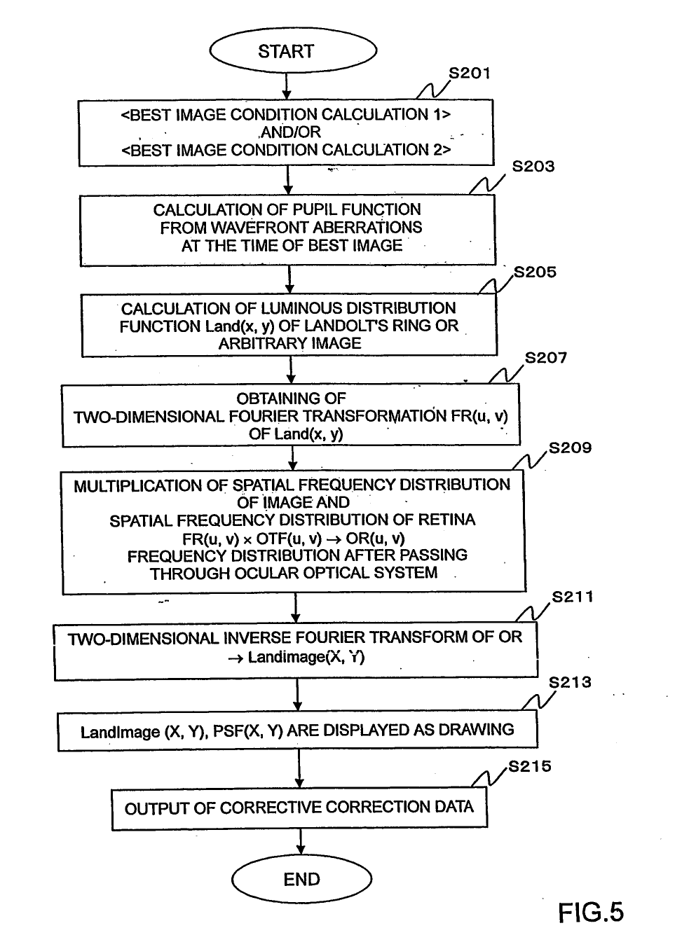

FIG. 5 is a flowchart of the correction image simulation of the step S107.

The arithmetic part 210 calculates a best image condition (S201). As described later, the details are such that the arithmetic part 210 obtains a lower order Zernike coefficient so that the Strehl ratio becomes maximum or the phase shift becomes as small as possible, and obtains corrective correction data. As the corrective correction data, suitable data can be named among, for example, coefficients corresponding to defocus, astigmatism components, S, C, A, higher order spherical aberrations, higher order astigmatism aberrations, higher order coma aberrations, the Strehl ratio and the like.

The arithmetic part 210 obtains the wavefront aberrations W(x, y) at the time of the best image condition, and calculates the pupil function f(x, y) from W(x, y) by the following expression (S203).

$$W ( X , Y ) = {\sum }_{i = 0}^{n} {\sum }_{j = 0}^{i} {c}_{i}^{2 j - i} {Z}_{i}^{2 j - i} ( X , Y ) ( i ≧ 1 , 1 ≦ j ≦ i )$$

$$f ( x , y ) = {ⅇ}^{ikW ( x , y )}$$

The arithmetic part 210 calculates the luminous distribution function Land(x, y) of the Landolt's ring (or arbitrary image) with reference to the memory 240 (S205). The arithmetic part 210 performs a two-dimensional Fourier transformation to obtain a spatial frequency distribution FR(u, v) (S207). The arithmetic part 210 obtains a frequency distribution OR(u, v) after passing through the ocular optical system by multiplying the spatial frequency distribution FR(u, v) of the Landolt's ring (or arbitrary image) and an ocular spatial frequency distribution OTF(u, v) together as indicated by the following expression (S209).
FR(u, v)×OTF(u, v)→OR(u, v)

Next, the arithmetic part 210 performs a two-dimensional inverse Fourier transform to obtain a luminous distribution image LandImage (X, Y) of the Landolt's ring (or arbitrary image) (S211). The arithmetic part 210 displays the LandImage(X, Y) and PSF(X, Y) on the display part 230 by a suitable display method of a drawing, graphic data, a graph and/or a numerical value, and suitably stores the data in the memory 240 (S213). The arithmetic part 210 reads but corrective correction data from the memory 240 as the need arises, and outputs it to the display part 230 (S215).

Hereinafter, a first and a second examples of a detailed flowchart concerning the step S201 will be described.

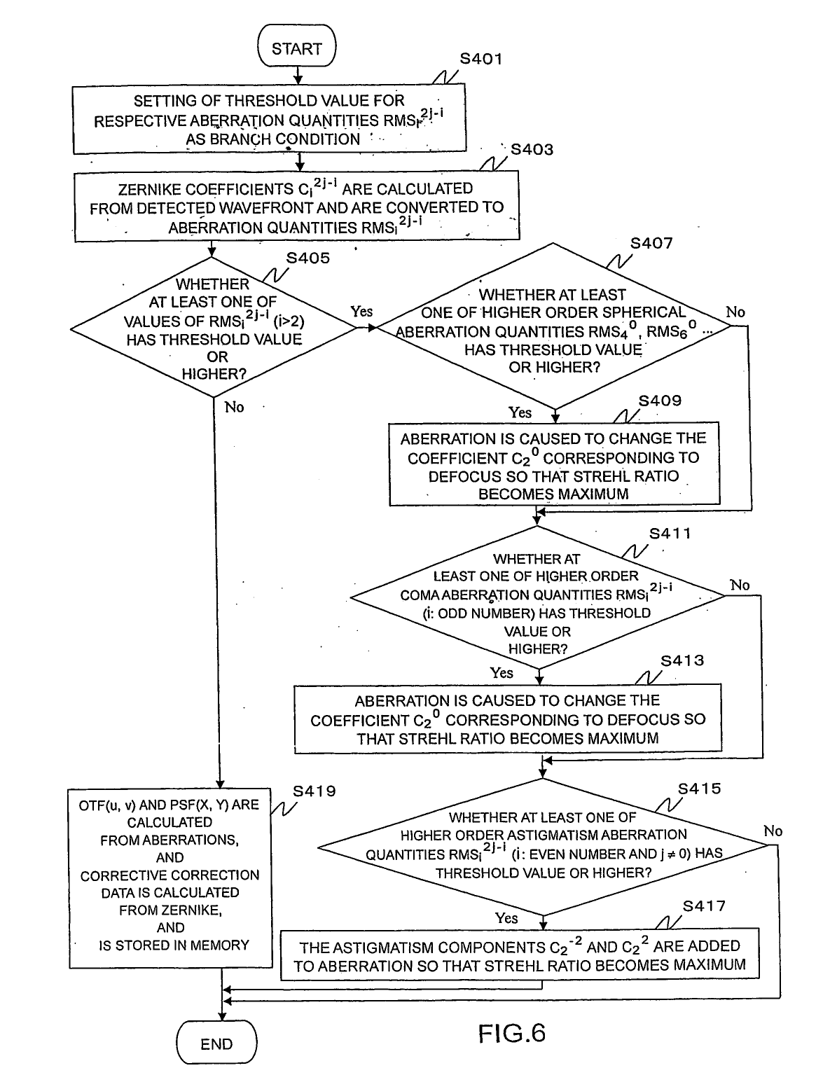

FIG. 6 shows the flowchart concerning the first example of the best image condition calculation.

First, the arithmetic part 210 sets a threshold value for respective aberration quantities RMSᵢ^{2j-i} as a branch condition (S401). For example, this threshold value can be made a sufficiently small value (for example, 0.1) of aberration. The arithmetic part 210 calculates the Zernike coefficients Cᵢ^{2j-i} from the measured detection wavefront, and converts them to the aberration quantities RMSᵢ^{2j-i} by the following expression (S403).

$${RMS}_{i}^{2 j - i} = \sqrt{\frac{{ɛ}_{i}^{2 j - 1}}{2 ( i + 1 )} {C}_{i}^{2 j - i}}$$

$$( {ɛ}_{i}^{2 j - i} = 2 ( 2 j = i ) , {ɛ}_{i}^{2 j - i} = 1 ( 2 j \neq i ) )$$

The arithmetic part 210 judges whether at least one of the values of RMSᵢ^{2j-i} (i>2) is the threshold value or higher (S405). Here, in the case where a judgment of No is made, it proceeds to step S419. On the other hand, here, when a judgment of Yes is made, the arithmetic part 210 carries out a next processing.

That is, the arithmetic part 210 judges whether at least one of the higher order spherical aberration quantities R₄⁰, R₆⁰ . . . is the threshold value or higher (S407). Here, in the case of Yes, the arithmetic part 210 causes the aberration to change a coefficient (C₂⁰) corresponding to the defocus so that the Strehl ratio becomes maximum (S409), and on the other hand, in the case of No, it proceeds to step S411. Next, the arithmetic part 210 judges whether at least one of the asymmetrical higher order coma aberration quantities RMSᵢ^{2j-i} (i: odd number) is the threshold value or higher (S411). Here, in the case of Yes, the arithmetic part 210 causes the aberration to change the coefficient (C₂⁰) corresponding to the defocus so that the Strehl ratio becomes-maximum (S413), and on the other hand, in the case of No, it proceeds to step S415. Further, the arithmetic part 210 judges whether at least one of the higher order astigmatism aberration quantities RMSᵢ^{2j-i} (i: even number and 2j−1≠0) is the threshold value or higher (S415). Here, in the case of Yes, the arithmetic part 210 adds astigmatism components (C₂⁻², C₂²) to the aberration so that the Strehl ratio becomes maximum (S417), and on the other hand, in the case of No, it proceeds to step S419.

In this way, the arithmetic part 210 calculates OTF(u, v) and PSF(X, Y) from the aberrations, and further calculates the corrective correction data (suitable data such as coefficients corresponding to the defocus, astigmatism components, S, C, A, higher order spherical aberrations, higher order astigmatism aberrations, higher order coma aberrations, and Strehl ratio) from the Zernike coefficients, and stores them in the memory 240 (S419).

Incidentally, in order to correct only a desired component among the defocus and the astigmatism components, any of the pairs of the steps S407 and S409, the steps S411 and S413, and the steps of S415 and S417 may be omitted, or a step may be added to correct suitable higher order aberrations or Zernike coefficients other than these. For example, in the case where a fourth-order spherical aberration is mainly included in the higher order aberrations, the corrective correction data can be obtained by correcting in the direction in which the defocus quantity corresponding to the lower order aberrations are increased.

Next, the detailed processing of the steps S409, S413 and S417 will be described. In the respective steps, the arithmetic part 210 carries out the processing as follows.

The wavefront at the time of the objective complete correction calculated from the Zernike coefficients is expressed by the following expression.

$$W ( X , Y ) = {\sum }_{i = 0}^{n} {\sum }_{j = 0}^{i} {c}_{i}^{2 j - i} {Z}_{i}^{2 j - i} ( X , Y ) ( i ≧ 3 , 3 ≦ j ≦ i )$$

In order to obtain a more suitable image plane, the arithmetic part 210 adds to the wavefront aberrations W(x, y) the lower order Zernike coefficients Cᵢ^{2j-i}(1≦i≦2) at each step presently noted for the aberration quantities comparable to the higher order aberration quantities according to the threshold value of the last noted higher order aberration quantities (RMS₄⁰, RMS₆⁰ . . . ) in the flow. For example, C₂⁰ is added at the step S409; C₂⁰, at the step 413; and C₂⁻², C₂², at the step S417.

$$W ( X , Y ) = {\sum }_{i = 0}^{n} {\sum }_{j = 0}^{i} {c}_{i}^{2 j - i} {Z}_{i}^{2 j - i} ( X , Y ) ( i ≧ 2 , 2 ≦ j ≦ i )$$

Further, the pupil function f(x, y) is obtained from the wavefront aberrations in the manner described below.
f(x, y)=e^{ikW(x, Y)}

(i: imaginary number, k: wave number vector (2π/λ), λ: wavelength)

The arithmetic part 210 performs the Fourier transformation on this pupil function f(x, y), so that an amplitude distribution U(u, v) of a point image is obtained as in the following expression.

$$amplitude U ( u , v ) = \int {\int }_{- ∞}^{∞} f ( x , y ) exp [ - \frac{i}{R} \frac{2 π}{λ} ( ux + vy ) ] ⅆ x ⅆ y$$

(λ: wavelength
R: a distance from a pupil to an image point (retina)
(u, v): a coordinate value on a plane orthogonal to an optical axis while an image point O is made the origin
(x, y): a coordinate value on a pupil plane)

The arithmetic part 210 multiplies U(u, v) by its complex conjugate and obtains I(u, v) as a point image intensity distribution (PSF) by the following expression.
I(u, v)=U(u, v)U*(u, v)

Besides, when the center intensity of PSF at the time when there is no aberrations (W(x, y)=0) is made Io(0, 0), the Strehl ratio is defined as follows:
Strehl ratio=I(0, 0)/Io(0, 0).

In the first example, the arithmetic part 210 recursively or analytically obtains a value of the lower order Zernike coefficient Cᵢⱼ (1≦i≦2) so that the value of the Strehl ratio becomes maximum.

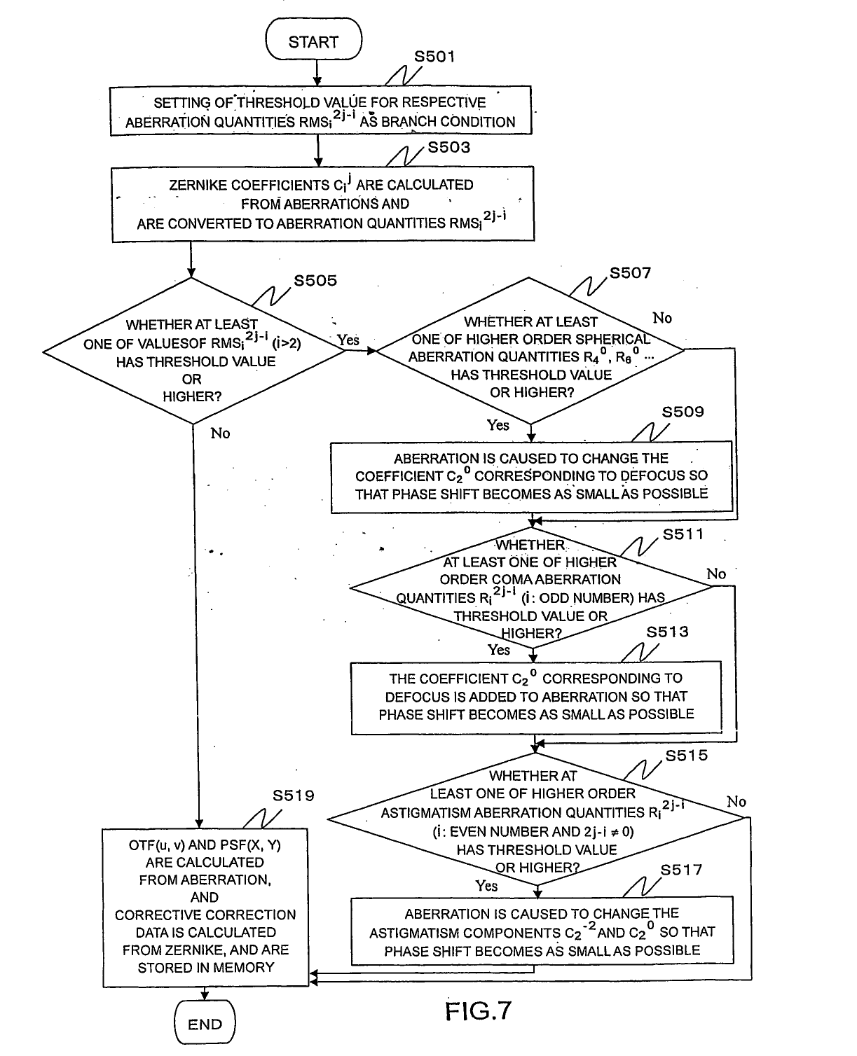

Next, FIG. 7 is a flowchart concerning the second example of the best image condition calculation.

First, the arithmetic part 210 sets a threshold value for the respective aberration quantities RMSᵢ^{2j-i} as a branch condition (S501). For example, this threshold value is made a sufficiently small value (for example, 0.1) of aberration.

The arithmetic part 210 calculates the Zernike coefficients Cᵢ^{2j-i} from the measured detection wavefront, and converts them to the aberration quantities RMSᵢ^{2j-i} by the expression indicated in the first example (S503). The arithmetic part 210 judges whether at least one of RMSᵢ^{2j-i} (i>2) is the threshold value or higher (S505). Here, in the case of the judgment of No, it proceeds to step S519. On the other hand, in the case of the judgment of Yes, the arithmetic part 210 carries out a next processing.

That is, the arithmetic part 210 judges whether at least one of the higher order spherical aberration quantities R₄⁰, R₆⁰ . . . is the threshold value or higher (S507). Here, in the case of Yes, the arithmetic part 210 causes the aberration to change the coefficient (C₂⁰) corresponding to the defocus so that the phase shift becomes as small as possible (S509), and on the other hand, in the case of No, it proceeds to step S511. Next, the arithmetic part 210 judges-whether at least one of the higher order coma aberration quantities RMSᵢ^{2j-i} (i: odd number) is the threshold value or higher (S511). Here, in the case of Yes, the arithmetic part 210 causes the aberration to change the coefficient (C₂⁰) corresponding to the defocus so that the phase shift becomes as small as possible (S513), and on the other hand, in the case of No, it proceeds to step S515. Further, the arithmetic part 210 judges whether at least one of the higher order astigmatism aberration quantities RMSᵢ^{2j-i} (i: even number and j≠0) is the threshold value or higher (S515). Here, in the case of Yes, the arithmetic part 210 adds the astigmatism components (C₂⁻², C₂²) to the aberration so that the Strehl ratio becomes maximum (S517), and on the other hand, in the case of No, it proceeds to step S519.

In this ways, the arithmetic part 210 calculates OTF(u, v) and PSF(X, Y) from the aberrations, and further calculates the corrective correction data (suitable data such as the coefficients corresponding to the defocus, astigmatism components, S, C, A, higher order spherical aberrations, higher order astigmatism aberrations, higher order coma aberrations, and Strehl ratio) from the Zernike coefficients, and stores them in the memory 240 (S519).

Incidentally, any of the pairs of the steps S507 and S509, the steps S511 and S513, and the steps S515 and S517 may be omitted so that only a desired component is corrected among the defocus and the astigmatism components. Besides, a step may be added so that suitable higher order aberrations or Zernike coefficients other than these is corrected.

Next, the detailed processing of the steps S509, S513 and S517 will be described. The arithmetic part 210 carries out the processing as follows.

First, as described in the detailed processing of the steps S409, S413 and S417, the arithmetic part 210 obtains the point image intensity distribution (PSF) from the expression of the wavefront at the time of the objective complete correction calculated from the Zernike coefficients. Next, the arithmetic part 210 performs a Fourier transformation (or autocorrelation) on the PSF to normalize it as in the following expression and obtains OTF.

$$R ( r , s ) = \int {\int }_{- ∞}^{∞} I ( u , v ) {ⅇ}^{- i2π ( ru + sv )} ⅆ u ⅆ v \\ ( r , s : a variable of spatial frequency region )$$

$$OTF = \frac{R ( r , s )}{R ( 0 , 0 )}$$

In general, the amplitude of a spatial frequency region and a phase distribution R(r, s) become complex numbers, and when its real number part is A(r, s), and its imaginary part is B(r, s),
R(r, s)=A(r, s)+iB(r, s)

and the shift of the phase (phase shift, PTF) is calculated by

$$ϕ ( r , s ) = {tan}^{- 1} \frac{B ( r , s )}{A ( r , s )}$$

In the second example, the arithmetic part 210 recursively and analytically obtains such a value of the lower order Zernike coefficient Cᵢ^{2j-i} that a value at which the R(r, s) has an extreme value is brought to a high frequency to the extent possible, that is, the phase shift becomes as small as possible.

Incidentally, with respect to the first example and the second example of the best image condition calculation, both the processings may be carried out to obtain such a condition that the Strehl ratio is large, and the phase shift is small.

5. Display Example

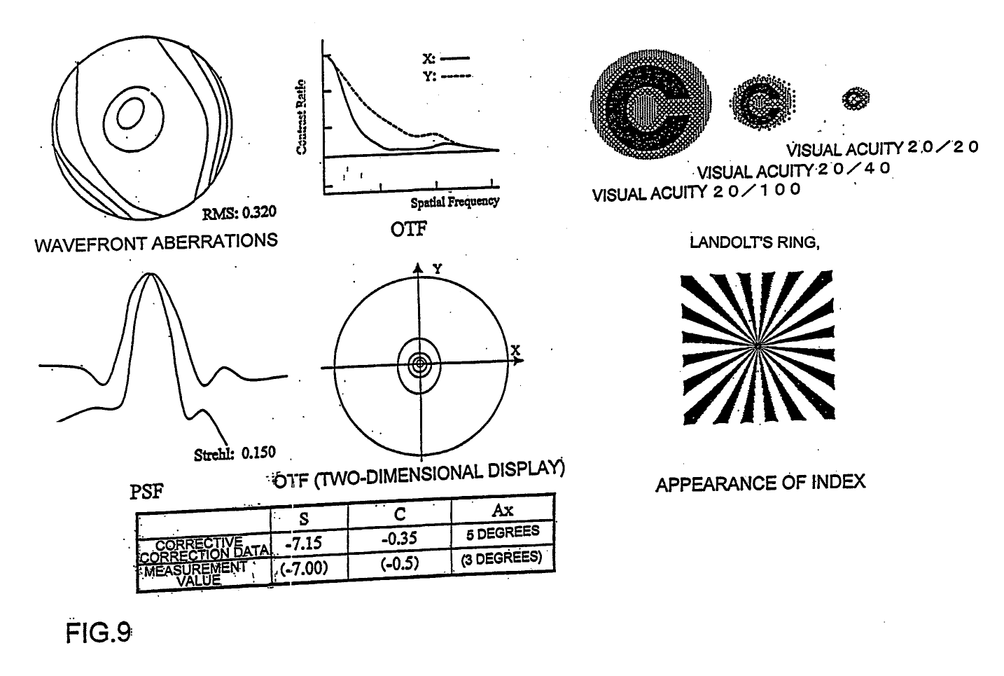

With respect to the best image display—Strehl ratio optimization (pupil diameter of 4 mm), FIG. 9 displays, as numerical data, sphere S, cylinder C, astigmatism axis angle Ax of the corrective correction data, and sphere S, cylinder C, and astigmatism axis angle Ax of measurement values before corrective correction. In this example, since higher order aberration components have specified values or higher, there occurs a difference between the corrective correction data and the measurement values.

With respect to the best image display—PTF optimization (pupil diameter of 4 mm), FIG. 9 displays, as numerical data, sphere S, cylinder C, and astigmatism axis angle Ax of corrective correction data, and sphere S, cylinder C, and astigmatism axis angle Ax of measurement values before corrective correction. In this example, since a higher order aberration components have specified values or higher, there occurs a difference between the corrective correction data and the measurement values.

In these drawings, the wavefront aberrations, PSF, OTF, OTF (two-dimensional display), S, C, Ax, Landolt's ring, appearance of index, and the like are displayed on the display part.

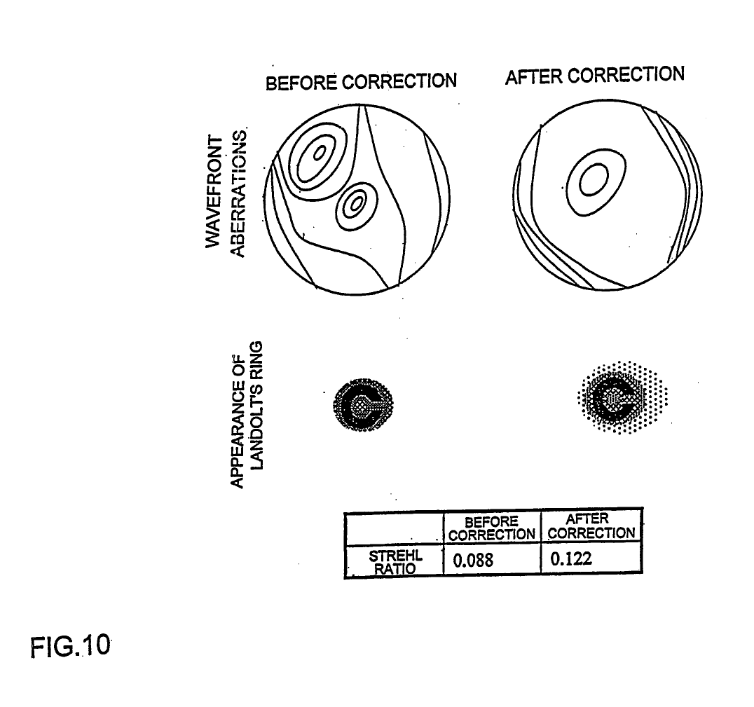

FIG. 10 is a view showing a display example concerning the comparison (pupil diameter of 4 mm) of pre-correction and post-correction. In this drawing, the wavefront, the appearance of the Landolt's ring, and the Strehl ratio before the correction and those after the correction are displayed. As shown in the drawing, it is indicated that after the correction, the Strehl ratio is higher, the wavefront aberrations become relatively uniform, and the Landolt's ring is also relatively well seen.

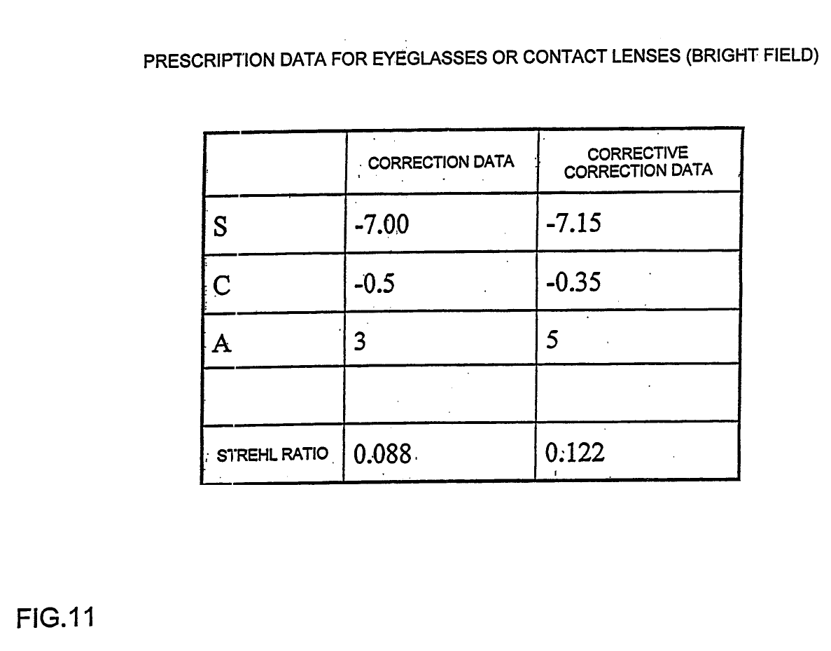

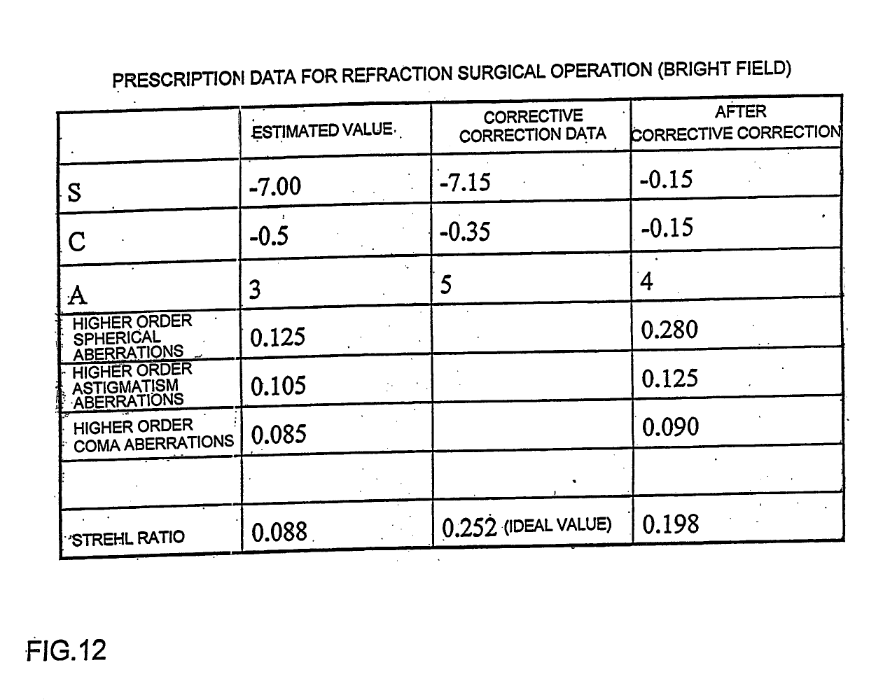

FIG. 11 is an explanatory view of an example of prescription data (pupil diameter of 4 mm) for eyeglasses or contact lenses. FIG. 12 is an explanatory view of an example of data (pupil diameter of 4 mm) for a refraction correcting surgical operation.

These data are stored in the memory 240 by the arithmetic part 210, and/or displayed on the display part 230. This example indicates that in the data of a case where the refraction correcting surgical operation is performed while only SCA are made correction data, by performing correction to intensify the value of S in the correction data, to weaken the value of C, and to slightly change the axial direction of A, the Strehl ratio becomes high and the correction effect becomes high.

A modified example of the invention will be described below.

This modified example modifies the calculation method of the best image condition at S201 of FIG. 5.

A component of an i-th row and a j-th column of Jacobian matrix A is

$${A}_{ij} = \frac{\partial {f}_{i} ( x )}{\partial {x}_{j}} \qquad ( 1 )$$

Where, fᵢ(x) is the Strehl ratio, the PTF corresponding to a suitable frequency, or some values of the PTF corresponding to plural frequencies. Besides, it may be a combination of the Strehl ratio and the PTF. Besides, a vector x is an adjustable parameter, and here, the sphere (or defocus corresponding to that) and two astigmatisms correspond to that.

The calculation expressions of the Strehl ratio and the PTF are already given. The ideal value of the. Strehl ratio is 1. It is assumed that the following expression expresses the Strehl ratio.
f₁(x)=fs(hc, c₂⁰, c₂⁻², c₂²) (2)

Where, f₁ denotes the expression of the same indication in the-expression (1).

Besides, for example, as the PTF, values corresponding to the spatial frequency-of 3 cpd, 6 cpd, 12 cpd, and 18 cpd are taken, and it is ideal that this is 0.
f₂(x)=f_{PTF3}(hc, c₂⁰, c₂⁻², c₂²) (3)
f₃(x)=f_{PTF6}(hc, c₂⁰, c₂⁻², c₂²) (4)
f₄(x)=f_{PTF12}(hc, c₂⁰, c₂⁻², c₂²) (5)
f₅(x)=f_{PTF18}(hc, c₂⁰, c₂⁻², c₂²) (6)

In the expressions (2), (3), (4), (5) and (6), hc denotes a vector of higher order aberration coefficients, c₂⁰ denotes a coefficient of a defocus term relating to the sphere, c₂⁻² and c₂² denote coefficients of terms relating to astigmatism. The vector hc is given by wavefront aberration measurement, and here it is constant. Thus, the remaining three coefficients are made a parameter vector x and are suitably moved to guide fₛ to the minimum value, which is a task here.

Here, the partial differentiation of the expression 1 can be calculated by slightly moving the parameters to prepare a change table, and the Jacobian matrix in this system is obtained.

Now, when the task here is expressed in other words again, since nonlinear optimization in the case where the Jacobian, that is, the partial differential coefficient is known has only to be performed, when optimizing algorism of a Newton method system is used, it is easy to obtain a solution since the example is simple. When a specific solution according to a corrected Marquardt method is stated, a correction vector Δx can be obtained by
(A^{t}WA+λI)Δx=A^{t}W(y−f(x)) (7)

Here, t at the shoulder of the matrix denotes a transposed matrix, and W denotes a weighting matrix. The first element of y corresponds to Strehl ratio, and the remainder corresponds to four components of PTF, it has only to be made (1, 0, 0, 0, 0)^{t}. λ is called a damping factor, and it is made large at first, and then, it is made small in accordance with going of optimization.

$$W = ( \begin{matrix}{w}_{1} & 0 & 0 & 0 & 0 \\ 0 & {w}_{2} & 0 & 0 & 0 \\ 0 & 0 & {w}_{3} & 0 & 0 \\ 0 & 0 & 0 & {w}_{4} & 0 \\ 0 & 0 & 0 & 0 & {w}_{5}\end{matrix} ) \qquad ( 8 )$$

A subscript corresponds to a subscript of f. Weighting suitable for the object of a prescription can be freely performed, for example, when the Strehl ratio is desired to be selectively optimized, W₁ is made large. The expression (7) is applied several times, and when
S=W(y−f(x)) (9)

becomes suitably small (when a conversion condition is satisfied), the calculation is stopped, and x at that time is made the solution. By this, the optimum the sphere (or defocus corresponding to that) and two astigmatisms are obtained.

Incidentally, the best image condition calculation can also be performed by finding out the position where the Strehl ratio becomes maximum or the phase, shift (PTF) becomes substantially zero while the defocus amount and/or the astigmatism component is changed slightly. Besides, a position where the Strehl ratio becomes maximum or the phase shift (PTF) becomes substantially zero may be obtained by using a well-known Newton method.

6. Addition

The correction data measurement method of the invention or the apparatus and system of the correction data measurement can be provided by a correction data measurement program for causing a computer to execute the respective procedures, a computer readable recording medium recording the correction data measurement program, a program product including the correction data measurement program and capable of being loaded in an internal memory of a computer, a computer, such as a server, including the program, and the like.

Although the measurement data indicating the refractive power distribution of the subject eye is obtained by the optical system 100 shown in FIG. 1, the invention is not limited to this, but may be structured by another aberrometer or the like.

### INDUSTRIAL APPLICABILITY

As described above, according to the invention, from the result measured by the eye characteristic measuring apparatus capable of performing the measurement up to higher order aberrations, optical performance is evaluated by, for example, the Strehl ratio or the phase shift in not only the case of only the higher order aberrations corresponding to the objective complete correction, but also the case in which the lower order aberrations are added, such lower order aberration quantities that for example, the Strehl ratio is large and/or the phase shift becomes small is calculated, and the correction data of S, C, A and the like at that time are obtained, so that the result closer to the subjective value can be obtained.

1. A correction data measurement method comprising:
a first step at which an arithmetic part receives measurement data indicating a refractive power distribution of a subject eye and obtains lower order aberrations and higher order aberrations on the basis of the measurement data;
a second step at which the arithmetic part judges whether the higher order aberrations have specified values or higher; and
a third step at which the arithmetic part changes, in a case where the higher order aberrations have the specified values or higher, lower order aberration quantities corresponding to the higher order aberrations having the specified values or higher and obtains appropriate correction data suitable for the subject eye.

2. A correction data measurement method according to claim 1, wherein at the third step, in a case where higher order spherical aberrations or asymmetrical higher order coma aberration quantities have the specified values or higher, the arithmetic part changes the lower order aberration quantities corresponding to defocus, and obtains the appropriate correction data suitable for the subject eye.

3. A correction data measurement method according to claim 1, wherein at the third step, in a case where higher order astigmatism aberration quantities have the specified values or higher, the arithmetic part changes the lower order aberration quantities corresponding to astigmatism components, and obtains the appropriate correction data suitable for the subject eye.

4. A correction data measurement method according to claim 2, wherein at the third step, the arithmetic part changes the lower order aberration quantities to raise a Strehl ratio, and obtains the appropriate correction data suitable for the subject eye.

5. A correction data measurement method according to claim 2, wherein at the third step, the arithmetic part changes the lower order aberration quantities to decrease a phase shift, and obtains the appropriate correction data suitable for the subject eye.

6. A correction data measurement method according to claim 1, further comprising a fourth step of storing the correction data obtained by the arithmetic part into a memory or displaying it on a display part.

7. A correction data measurement method according to claim 6, wherein at the fourth step, on the basis of the correction data obtained by the arithmetic part, a luminous distribution image of a Landolt's ring or an arbitrary image is obtained, and is displayed on a display part.

8. A computer readable recording medium recording a correction data measurement program for causing a computer to execute:
a first step at which an arithmetic part receives measurement data indicating a refractive power distribution of a subject eye and obtains lower order aberrations and higher order aberrations on the basis of the measurement data;
a second step at which the arithmetic part judges whether the higher order aberrations have a specified values or higher; and
a third step at which the arithmetic part changes, in a case where the higher order aberrations have the specified values or higher, lower order aberration quantities corresponding to the higher order aberrations having the specified value or higher and obtains appropriate correction data suitable for the subject eye.

9. A correction data measurement program for causing a computer to execute:
a first step at which an arithmetic part receives measurement data indicating a refractive power distribution of a subject eye and obtains lower order aberrations and higher order aberrations on the basis of the measurement data;
a second step at which the arithmetic part judges whether the higher order aberrations have a specified values or higher; and
a third step at which the arithmetic part changes, in a case where the higher order aberrations have the specified value or higher, lower order aberration quantities corresponding to the higher order aberrations having the specified value or higher and obtains appropriate correction data suitable for the subject eye.

10. A correction data measuring apparatus comprising:
an arithmetic part for obtaining an optical characteristic of a subject eye by performing a Zernike analysis on the basis of inclination angles of light fluxes obtained by a first light receiving part, wherein
the arithmetic part includes:
first means for receiving measurement data indicating a refractive power distribution of the subject eye and obtaining lower order aberrations and higher order aberrations on the basis of the measurement data;
second means for judging whether the higher order aberrations have a specified values or higher, and
third means for changing, in a case where the higher order aberrations have the specified values or higher, lower order aberration quantities corresponding to the higher order aberrations having the specified values or higher and obtaining appropriate correction data suitable for the subject eye.

11. A correction data measuring apparatus according to claim 10, wherein in a case where higher order spherical aberrations or asymmetrical higher order coma aberration quantities have the specified value or higher, in the third means, the arithmetic part changes the lower order aberration quantities corresponding to defocus, and obtains the appropriate correction data suitable for the subject eye.

12. A correction data measuring apparatus according to claim 10, wherein in a case where higher order spherical aberration quantities have the specified values or higher, in the third means, the arithmetic part changes the lower order aberration quantities corresponding to astigmatism components, and obtains the appropriate correction data suitable for the subject eye.

13. A correction data measuring apparatus according to claim 11, wherein in the third means, the arithmetic part changes the lower order aberration quantities to increase a Strehl ratio and obtains the appropriate correction data suitable for the subject eye.

14. A correction data measuring apparatus according to claim 11, wherein in the third means, the arithmetic part changes the lower order aberration quantities to decrease a phase shift and obtains the appropriate correction data suitable for the subject eye.

15. A correction data measuring apparatus according to claim 10, further comprising fourth means for storing the correction data obtained by the arithmetic part in a memory or displaying it on a display part.

16. A correction data measuring apparatus according to claim 15, wherein the fourth means obtains a luminous distribution image of a Landolt's ring or an arbitrary image on the basis of the correction data obtained by the arithmetic part and displays it on the display part.

17. A correction data measuring apparatus according to claim 10, further comprising:
a first illuminating optical system including a first light source for emitting a light flux of a first wavelength, for providing illumination by condensing a first illuminating light flux from the first light source on a vicinity of a retina of the subject eye; and
a first light receiving optical system including a first conversion member for converting a reflected light flux reflected from the retina of the subject eye into at least 17 beams, and a first light receiving part for receiving the plural light fluxes converted by the first conversion member as a first received light signal, for guiding the reflected light flux to the first light receiving part,
wherein the arithmetic part receives the first received light signal as the measurement data, and performs the Zernike analysis on the basis of the inclinations of the light fluxes obtained by the first light receiving part to obtain the lower order aberrations and the higher order aberrations as the optical characteristic of the subject eye.

18. A correction data measurement method comprising steps of:
receiving measurement data indicating a refractive power distribution of a subject eye and obtaining lower order aberrations and higher order aberrations on the basis of the measurement data;
forming a relational expression of a Strehl ratio and a phase shift (PTF) from the obtained lower order aberrations and higher order aberrations; and
changing the lower order aberrations to obtain a condition under which the Strehl ratio becomes maximum and the phase shift (PTF) becomes substantially zero, and making lower order aberration quantities at that time a correction value.
